(19) United States
(12) Patent Application Publication
(10) Pub. No.: US 2002/0198420 A1
(43) Pub. Date: Dec. 26, 2002

(54) Mono-alkylation process for the preparation of anionic surfactants

(75) Inventors: Giao Vinh Nguyen, Friendswood, TX (US); James Alfred Ragains, Houston, TX (US)

Correspondence Address: Kenneth D. Tremain, UNIROYAL CHEMICAL COMPANY, INC., World Headquarters, Middlebury, CT, 06749, US

(73) Assignee: CROMPTON CORPORATION.

(21) Appl. No.: 09/812,921

(22) Filed: Mar. 20, 2001

(51) Int. Cl. C07C 15/067

(52) U.S. Cl. 585/446

(57) ABSTRACT

A process for mono-alkylating monocyclic aromatic hydrocarbons is disclosed wherein the process comprises reacting at least one monocyclic aromatic hydrocarbon with at least one α-olefin having from 4 to 20 carbon atoms in the presence of an anhydrous alkane sulfonic acid at a temperature below about 280° F.

### BACKGROUND OF THE INVENTION

1. Field of the Invention

The present invention relates to the mono-alkylation of monocyclic aromatic hydrocarbons. More particularly, the present invention relates to the mono-alkylation of phenyl rings, preferably alkyl substituted benzene rings, such as toluene or xylene, with α-olefins having from 4 to 20 carbon atoms.

2. Description of Related Art

Anionic surfactants, particularly of the alkylarene sulfonate type, have been used as oil-soluble or water-soluble emulsifiers in several applications. Two of the most important applications for these emulsifiers are in metal working and enhanced oil recovery (EOR).

Typically, alkylarene sulfonates are prepared by a three-step process:

alkylation of a selected aromatic hydrocarbon (arene) with a selected olefin to form an alkylarene or “alkylate”,

sulfonation of the resulting alkylarene (“alkylate”), preferably with sulfur trioxide, to yield the alkylarene sulfonic acid, and

neutralization of the alkylarene sulfonic acid with a desired base (sodium hydroxide, potassium hydroxide, amines, and the like) to yield the final product, an anionic surfactant.

Of these three steps, it is the alkylation step that determines the performance of the resulting anionic surfactant. In this step, the lipophilic-hydrophilic balance of the surfactant, and, hence, its emulsification property, is determined by the selection of the proper molecular size of the alkylating agent (the olefin).

Typically, the alkylation, commonly known as the Friedel-Crafts reaction, involves a reaction between an aromatic hydrocarbon and an alkyl halide that is catalyzed by a Lewis acid, such as aluminum chloride or hydrogen fluoride. The most characteristic property of a Friedel-Crafts reaction is polyalkylation, which occurs when the freshly formed alkylarene, itself, undergoes alkylation, i.e.,

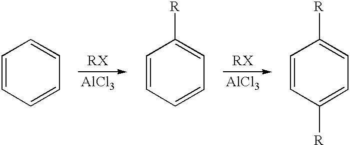

This polyalkylation is of concern, since it results in several adverse effects with regard to the alkylarene sulfonates, such as:

It necessitates the use of an excess quantity of the aromatic hydrocarbon. As a result, costly distillation is required to remove the excess aromatic from the alkylate mixture.

If excess aromatic cannot be used, it leaves a significant quantity of the starting aromatic in the alkylate mixture.

Both the unreacted aromatic and the dialkylarene (from the polyalkylation) are readily reactive toward the sulfonation. The products of their sulfonation strongly and adversely affect the performance of the desired alkylarene sulfonates.

U.S. Pat. No. 3,959,399 discloses the inhibition of polyalkyl and specially dialkyl naphthalenes in the alkylation of naphthalene using an alkene reactant to produce monoalkyl naphthalene, by the use of a mixed protonic acid catalyst consisting of methane sulfonic acid and an active P₂O₅ containing acid, utilized in about a 2:1 to 1:2 ratio with an optimum ratio of about 1:1. The reaction is carried out preferably under anhydrous conditions with respect to the mixed catalyst, and the products are said to show utility as emulsion breakers in petroleum chemistry as well as other surface active agents.

U.S. Pat. No. 4,482,755 discloses the production of 4,4′-biphenol and tert-alkyl substituted alkyl benzene derivatives by hydrogenating a tetraalkyl diphenoquinone in an alkyl benzene solvent solution under relatively mild conditions in the presence of a heterogeneous catalyst, removing the catalyst from the resultant tetraalkyl biphenol and thereafter heating the alkyl benzene solvent solution in the presence of a strong acid catalyst to form relatively pure biphenol in high yield and a para substituted alkyl benzene derivative.

U.S. Pat. No. 5,889,137 discloses the formation of phenol alkylation polymers which release negligible phenol and formaldehyde emissions. The phenol alkylation polymers are derived from a phenolic monomer, at least one styrene derivative and an aryl diolefin. In addition to the phenolic monomer, styrene derivative and aryl diolefin, other reactants may be introduced to produce a product with particular properties.

U.S. Pat. No. 6,043,391 discloses anionic surfactants, and methods for their preparation, that are derived from aromatic or substituted aromatic molecules and alkenesulfonic acid. The aryl compound is alkylated and sulfonated in one-step with an alkene sulfonic acid prior to sulfonic acid neutralization. The methods are said to allow the functional sulfonate group to be attached to the end of the alkyl chain rather than to the aromatic ring thus allowing for selective substituted groups, either branched, linear or alkoxylated or combinations thereof to be placed on the aryl compound prior to sulfonation and alkylation. The invention uses the alkene sulfonic acid produced from thin-film sulfonation of an alpha-olefin to alkylate benzene, mono-substituted aromatic, poly-substituted aromatic, alkylbenzene, alkoxylated benzene, polycyclic aromatic, mono-substituted polycyclic aromatic, poly-substituted polycyclic aromatic, naphthalene, alkylnaphthalene, phenol, alkylphenol, alkoxylated phenol, and alkoxylated alkylphenolalkyl substituted or polysubstituted cyclic or polycyclic compounds to produce the corresponding sulfonic acid having an additional alkyl group derived from the alpha-olefin used during the thin-film sulfonation which is either linear or branched.

Published European Patent Application 0 121 964 A1 discloses alkyl aryl sulfonate concentrate compositions and provides a process wherein an aqueous solution containing at least 10% w/w of a neutralizing agent is mixed with at least one C₂₋₉ saturated alcohol and the resulting mixture is used to neutralize a C₈₋₁₈ alkyl aryl (xylene or toluene) sulfonic acid, relative quantities being such that the resulting neutralized mixture contains 5 to 40 parts by weight of the alcohol per 100 parts by weight of alkyl aryl sulfonate salt. The resulting flowable liquid concentrate compositions are said to be easily handled materials having application in enhanced oil recovery processes.

U.S. Pat. No. 3,959,399, supra, refers to George A. Olah, Friedel-Crafts and Related Reactions, Vol. 2, Part 1. 1964, Interscience-Wiley, pages 1-31; 69-71; and 180-186. Inter alia, this reference discusses the cationic nuclear alkylation of various aromatics such as monocyclic and polycyclic hydrocarbons, phenols, amines, thiophenes, furans, etc., with simple olefins including the aryl-substituted olefins, styrene, and allylbenzene, etc. In the paragraph bridging pages 24 and 25, it is disclosed that strong protonic acids are very effective catalysts for the reaction of olefins with aromatics. Sulfuric acid, phosphoric acid, alkanesulfonic acids, and hydrogen fluoride are said to be effective catalysts for the reaction of benzene with propene.

The disclosures of the foregoing are incorporated herein by reference in their entirety.

### SUMMARY OF THE INVENTION

The present invention relates to the mono-alkylation of monocyclic aromatic hydrocarbons, preferably alkyl substituted benzene rings, such as toluene or xylene, with α-olefins having from 4 to 20 carbon atoms.

More particularly, the present invention is directed to a process for mono-alkylating at least one monocyclic aromatic hydrocarbon comprising reacting the monocyclic aromatic hydrocarbon with at least one α-olefin having from 4 to 20 carbon atoms in the presence of an anhydrous alkane sulfonic acid at a temperature below about 280° F. (about 138° C.). Preferably, the temperature employed for the alkylation is in the range of from about 180° F.(about 82° C.) to about 280° F. (about 138° C.) , more preferably, about 200° F. (about 93° C.) to about 275° F. (about 135° C.), most preferably, about 250° F. (about 121° C.) to about 270° F. (about 132° C.)

### DESCRIPTION OF THE PREFERRED EMBODIMENTS

As stated above, the present invention is directed to a process for mono-alkylating a monocyclic aromatic hydrocarbon comprising reacting the monocyclic aromatic hydrocarbon with an α-olefin having from 4 to 20 carbon atoms in the presence of an anhydrous alkane sulfonic acid at a temperature below about 280° F.

In a conventional Friedel-Crafts alkylation process, the preparation of a monoalkylarene is begun with the alkylation step, in which the alkylating agent (“olefin”) is reacted with the aromatic hydrocarbon (“aromatic”) to yield a mixture of unreacted (“excess”) aromatic, the desired monoalkylarene, and undesired dialkylarene. The aluminum chloride catalyst is not very effective, and thus is usually used in combination with a catalyst booster, a charge transfer compound, such as nitromethane, to boost its effectiveness.

In the next two steps, the reaction mixture is washed, first with a diluted sodium hydroxide solution, then with water to remove the catalyst and the catalyst booster from the alkylate mixture (aromatic, monoalkylarene, and dialkylarene). Upon separation, the wash layers are removed and must be disposed of properly as hazardous waste.

The alkylate mixture is then dried to remove water that has been dispersed in the mixture. Finally, to obtain monoalkylarene with high purity (95% or higher), the alkylate mixture is fractionally distilled. The first fraction is the unreacted aromatic, which is recycled to the next batch, and the second fraction is the desired monoalkylarene. Typically, the undesired dialkylarene is not distilled off and remains in the reactor as a dark colored, viscous material.

Conventional Friedel-Crafts alkylation is a complicated process; a typical process for the commercial production of the desired monoalkylarene may include up to 25 steps. To accommodate this process, complicated equipment is required. In addition to the alkylation reactor, the process also requires a fractional distillation unit and separate storage tanks for aromatic, olefin, nitromethane, sodium hydroxide, monoalkylarene, dialkylarene, and hazardous waste.

On the other hand, the MSA-catalyzed modified Friedel-Crafts alkylation process of the present invention is very simple, involving only two steps. In the first step, the alkylating agent (“olefin”) is reacted with a stoichiometric quantity of the aromatic hydrocarbon (“aromatic”) in the presence of methane sulfonic acid (MSA). Upon completion of the reaction, the mixture is allowed to stand. The catalyst separates from the alkylate layer, and is removed and recycled to the next batch. The reaction yields predominantly monoalkylarene.

Since both the aromatic and olefin are mostly consumed in the reaction, their concentrations in the alkylate mixture are insignificant. Thus, fractional distillation is not necessary.

The equipment for the MSA-catalyzed alkylation process is also simple. Only the alkylation reactor and storage tanks for aromatic, olefin, catalyst, and recycled catalyst are required.

Any monocyclic aromatic hydrocarbon having a position on the phenyl nucleus available for acceptance of an α-olefin having from 4 to 20 carbon atoms can be employed in the practice of the present invention. Those skilled in the art will recognize that steric effects may preclude the reaction of certain combinations of substituted phenyl rings and bulky α-olefins, such as those having branches near the site of the double bond; however, a determination of likely pairs can be readily made by skilled chemists, either intuitively or by routine experimentation.

As employed herein, the term “monocyclic aromatic hydrocarbon” is intended to exclude aromatic hydrocarbons having fused aromatic rings, e.g., naphthalene, anthracene, phenanthrene, and the like, but is not intended to exclude those hydrocarbons that have more than one aromatic ring where such rings are not fused, e.g., biphenyl and the like.

The monocyclic aromatic hydrocarbons contemplated as one of the reactants in the method of the present invention include, but are not limited to, at least one of benzene, toluene, o-xylene, m-xylene, p-xylene, hemimellitene, pseudocumene, mesitylene, prehnitene, isodurene, pentamethylbenzene, ethylbenzene, n-propylbenzene, cumene, n-butylbenzene, isobutylbenzene, sec-butylbenzene, tert-butylbenzene, p-cymene, biphenyl, diphenylmethane, triphenylmethane, 1,2-diphenylethane, styrene, trans-stilbene, cis-stilbene, unsym-diphenylethylene, triphenylethylene, tetraphenylethylene, phenylacetylene, diphenylacetylene, and the like. Preferably, the monocyclic aromatic hydrocarbon employed in the practice of the present invention is selected from the group consisting of benzene, toluene, o-xylene, m-xylene, p-xylene and mixtures thereof More preferably, it is o-xylene and/or p-xylene; most preferably o-xylene.

In accordance with the present invention, the monocyclic aromatic hydrocarbon is alkylated by reaction with at least one α-olefin having from 4 to 20 carbon atoms, e.g., 1-butene, 1-pentene, 1-hexene, 1-heptene, 1-octene, 1-nonene, 1-decene, 1-undecene, 1-dodecene, 1-tridecene, 1-tetradecene, 1-pentadecene, 1-hexadecene, 1-heptadecene, 1-octadecene, 1-nonadecene, 1-eicosene, and isomers and mixtures thereof Preferably, the α-olefin will be selected from the group consisting of 1-decene, 1-dodecene, 1-tetradecene, 1-hexadecene, and 1-octadecene; more preferably, 1-dodecene, 1-tetradecene, or 1-hexadecene. Most preferably, the α-olefin employed in the practice of this invention is 1-dodecene.

The above-described monocyclic aromatic hydrocarbon(s) and α-olefin(s) are reacted together in the presence of an anhydrous alkane sulfonic acid catalyst. It is preferred that the alkyl moiety of the alkane sulfonic acid be one of from one to four carbon atoms, e.g., methyl, ethyl, propyl, butyl, or isomers thereof, e.g., isopropyl, sec-butyl, isobutyl, tert-butyl, and the like. The most preferred catalyst for use in the practice of the present invention is anhydrous methane sulfonic acid.

Various features and aspects of the present invention are illustrated further in the examples that follow. While these examples are presented to show one skilled in the art how to operate within the scope of the invention, they are not intended in any way to serve as a limitation upon the scope of the invention.

### EXAMPLES

### Example 1

Linear dodecyl-o-xylene was prepared by the catalyzed reaction between 1-dodecene and o-xylene. A modified Friedel-Crafts reaction was used for the alkylation. Anhydrous methane sulfonic acid (MSA) was employed rather than the typical Friedel-Crafts catalyst (AICl₃, BF₃, HF, etc.) for several reasons.

First, it is very effective, and the MSA-catalyzed alkylation is much easier to control.

Second, the alkylation process is much simpler—no water washing and subsequent dehydration of the alkylate is required.

Third, MSA can be removed easily from the reaction mixture and can be recycled several times before losing its catalytic ability.

Finally, and most important, the MSA-catalyzed alkylation does not enhance the formation of the dialkylate (didodecyl-o-xylene). Since dialkylarenesulfonic acids generally perform very poorly in metal working applications, a primary use, this minimized formation of the dialkylate in the MSA-catalyzed alkylation of the present invention is extremely important, not only with regard to the performance of the final product, but also to the simplification of the alkylation process.

The alkylation is performed by charging the reactants, 1-dodecene and o-xylene, and the catalyst, methane sulfonic acid, to a glass-lined reactor. The mixture is then agitated and heated to a temperature in the range of from about 180 to about 200° F. (about 82 to about 93° C.) to initiate the reaction. Once initiated, the alkylation is highly exothermic, and the temperature rises steadily. The temperature is maintained within an optimum range of from about 250° to about 270° F. (about 121 to about 132° C.) and it is necessary to employ cooling water to do this. Water at a temperature of about 150° F. (about 66° C.) has been found adequate for this purpose. Within one hour at this temperature, up to 95% of the α-olefin has been converted. Typically, the alkylation is completed within 2 hours of digestion at the above-described optimum temperature, i.e., about 250 to about 270° F. (about 121 to about 132° C.)

To separate and remove the catalyst, the reaction mixture is cooled to about 150° F. (about 66° C.) Agitation is then stopped, and the mixture is allowed to stand for 1-2 hours. The MSA catalyst separates as a brown, viscous layer in the bottom of the reactor and can be removed by drainage.

A slight excess of o-xylene (1.20 moles per 1.00 mole of 1-dodecene) can be used in this process, if desired. At this molar ratio, virtually all olefin is consumed. The alkylation results in less than 1% of unreacted dodecene in the final alkylate product. However, since unreacted xylene can also react with sulfonating agent in a subsequent sulfonation reaction and yield product that may adversely affect the performance of the final sulfonated product for some applications, it may be necessary to remove the excess o-xylene in the alkylate mixture. Distillation at a temperature in the range of from about 230 to about 250° F. (about 110 to about 121° C.) under nitrogen bubbling and/or vacuum can reduce the o-xylene content to less than 1.0%. However, such a distillation step requires additional equipment, a distillation system, and prolongs the cycle time. The alkylation process can be simplified further by the use of the stoichiometric quantity of o-xylene. In an experiment at this 1:1 molar ratio, the alkylation was only 98% completed. Up to 2% of o-xylene and 1.5% of 1-dodecene remained unreacted. However, it was found that the linear dodecylxylene sulfonic acid prepared from this “crude” linear dodecylxylene could perform acceptably as a surfactant in some, if not all, applications. Therefore, at these low concentrations, removal of unreacted o-xylene and 1-dodecene may not be necessary, depending upon the application.

Typically, when an excess quantity of o-xylene is used, the concentration of the dialkylate in the alkylate mixture is less than 3%. As expected, the concentration of the dialkylate increases when the stoichiometric quantity of o-xylene is used. However, it is normally still less than 5%, and this level has not been found to have a negative effect on the performance of the sulfonate derivative.

Finally, it is important to note that when the reaction mixture containing the newly formed linear dodecyl-o-xylene is exposed to high temperature, two things will happen. The first is the decomposition of the methane sulfonic acid, which results in the loss of its catalytic ability. The second is the formation of branched alkylate that, upon sulfonation, yields product that may adversely affect the performance of the final product for some applications. For these reasons, it is important to maintain the alkylation temperature below about 280° F. (about 138° C.) at all times. See Table 1.

TABLE 1
Typical Compositions of the Linear Dodecylxylene Product Mixtures

| | Run #1 | Run #2 | Run #3 |
|---|---|---|---|
| Xylene/Olefin molar | 1.15:1.00 | 1.00:1.00 | 1.00:1.00 |
| ratio | | | |
| Alkylation | 250-270° F. | 250-270° F. | 300-330° F. |
| Temperature | | | |
| Composition: | | | |
| Free Xylene | <1.0% | 1.0-1.5% | 2.0-2.5% |
| Free Olefin | <1.0% | 1.0-1.5% | 1.5-2.0% |
| Dodecylxylene | >96.0% | 93.0-95.0% | 90.0-91.0% |
| Didodecylxylene | <2.5% | 2.5-3.5% | 3.0-4.0% |
| Branched Alkylate | — | <0.5% | 2.0-3.0% |

The alkylation process of the present invention provides the following advantages:

1. It utilizes monocyclic arenes (aromatic hydrocarbons with one ring), such as xylene and toluene, as the starting aromatic hydrocarbon. Since the steric hindrance and/or interaction are stronger in the monocyclic system than in a polycyclic system, the polyalkylation is reduced.

2. It utilizes methane sulfonic acid as the catalyst for the alkylation reaction. Unlike a standard Friedel-Crafts catalyst, e.g., aluminum trichloride, the methane sulfonic acid is not effective in catalyzing the alkylation of the newly formed alkylarene. Thus, a reduced amount of polyalkylated product is produced.

The combination of these two features permits the alkylation reaction to be carried out using stoichiometric quantities, or near-stoichiometric quantities, of reactants with minimum polyalkylation. The table below summarizes a comparison of the alkylation process of the present invention, wherein methane sulfonic acid is used as the catalyst, with a conventional

| | CONVENTIONAL | |
|---|---|---|
| | FRIEDEL-CRAFTS | PROCESS OF THE |
| PROPERTY | PROCESS | INVENTION |
| Starting Arene (Aro- | ο-Xylene | ο-Xylene |
| matic) | | |
| Alkylation Agent | 1-Dodecene | 1-Dodecene |
| (Olefin) | | |
| Olefin/Aromatic Molar | 1:1 | 1:1 |
| Ratio | | |
| Catalyst | Aluminum trichloride | Methane sulfonic acid |
| Product Composition: | | |
| Unreacted Olefin | <1.0% | 1-2% |
| Unreacted Aromatic | 5-6% | 1-2% |
| Alkylarene | 70-75% | >95% |
| Dialkylarene | 20-25% | <3% |

The above data show that the process of the present invention permits alkylarenes to be prepared using stoichiometric quantities of the aromatic hydrocarbon and alkylating agent (the olefin). The concentrations of the ureacted aromatic hydrocarbon, olefin, and, particularly, the dialkylarenes from the polyalkylation are low enough that removal is normally not necessary. This advantage permits a reduction of the cycle time of the process, which, in turn, reduces the total cost of the product.

### Example 2

### MSA versus Other Protonic Catalysts

In this series of experiments, the effectiveness of methane sulfonic acid and other protonic acids as catalysts for the alkylation of o-xylene and α-dodecene are compared. In each experiment, o-xylene (106 g, 1 mole), α-dodecene (168 g, 1 mole), and the tested catalyst were agitated in a glass reactor. The mixture was heated and held at 270° F. (132° C.) for several hours. Samples of the mixtures were then analyzed by gas chromatography (GC) to determine the olefin-alkylarene conversion as well as the concentration of the monododecyl-o-xylene and didodecyl-o-xylene. The results are summarized as in the following table.

| | Catalyst | | Olefin | Monododecyl-o- | Didodecyl-o- |
|---|---|---|---|---|---|
| | Conc. | Reaction | Conversion | xylene | xylene |
| Catalyst | (%) | Time (hrs) | (%) | (%) | (%) |
| MSA 99% | 7.5 | 2 | 99 | 95 | 3 |
| TX Acid⁽¹⁾ | 15.0 | 2 | 99 | 83 | 16 |
| TX Acid | 7.5 | 10 | 45 | 43 | 2 |
| H₂SO₄ 99%⁽²⁾ | 10.0 | 10 | 22 | 22 | — |
| H₃PO₄ 100% | 10.0 | 10 | 10 | 10 | — |
| DDBSA⁽³⁾ | 15.0 | 10 | <5 | <5 | — |

(1)TX Acid is a 1:1 blend of anhydrous toluene sulfonic acid and anhydrous xylene sulfonic acid.
(2)When sulfuric acid is used as catalyst, sulfonation of xylene with sulfuric acid is the predominant reaction. The alkylation is catalyzed by a combination of the resulting xylene sulfonic acid, and the remaining sulfuric acid.
(3)DDBSA is dodecylbenzene sulfonic acid.

The results of this series of experiments confirm that, when serving as the catalyst for the alkylation of aromatic with the α-olefin, the anhydrous methane sulfonic acid is superior to other common, commercially available protonic acids. Toluene sulfonic acid and toluene-xylene sulfonic acid (TX Acid) can also be an effective catalyst; however, it must be used at higher concentration and results in a higher concentration of the undesired dialkylxylene.

### Example 3

### Alkylation of Mixed Xylenes

The term “mixed xylenes” refers to a mixture of the three xylene isomers. Typically, mixed xylenes comprise o-xylene (about 25%), m-xylene (about 55%), and p-xylene (about 20%). In some applications, where the performance of the sulfonate derivatives is not significantly affected by the structure of xylene isomers, mixed xylenes are preferred over the high purity o-xylene owing to its low cost. In this example, MSA is shown to be equally effective in the alkylation of mixed xylenes with α-olefin.

Mixed xylenes (132.5 g, 1.2 moles), 1-dodecene (168 g, 1.0 mole), and methane sulfonic acid 99% (25 g, 8.3% batch weight) were agitated in a glass reactor. The mixture was then heated and held at 270° F. for 3 hours, when more than 99% of the olefin has been converted. Vacuum was then applied to distill off the excess mixed xylenes. The mixture was then cooled to 150° F. (650° C.) Agitation was stopped, and the mixture was allowed to stand for two hours. The catalyst, which separated from the alkylate product, was removed. GC analysis of the alkylate mixture showed that it contained mainly monododecylxylenes (93.5%), and that the concentration of the undesired didodecylxylenes remained low (4%)

### Example 4

### Alkylation with a Blend of α-Olefins

Ortho-xylene (106 g, 1 mole), 1-tetradecene (98 g, 0.5 mole), 1-hexadecene (112 g, 0.5 mole) and methane sulfonic acid 99% (27.5 g, 8.0% batch weight) were agitated in a glass reactor. The mixture was then heated and held at 270° F. for 3 hours, when more than 98% of the olefins had been converted. The mixture was then cooled to 150° F. (65° C.) Agitation was stopped, and the mixture was allowed to stand for two hours. The catalyst, which separated from the alkylate product, was removed. GC analysis of the alkylate mixture showed that it contained mainly monotetradecyl-/monohexadecylxylenes (92%), and that the concentration of the undesired dialkylxylenes remained low (3%).

### Example 5

### Alkylation with Branched Olefin

Branched olefins, particularly those prepared from the partial polymerization of propylene such as propylene trimers (branched nonene) and propylene tetramers (branched dodecene), can be used as the alkylating agent. This is advantageous owing to their lower cost.

Ortho-xylene (106 g, 1.00 mole), propylene tetramers (194 g, 1.15 moles), and methane sulfonic acid 99% (25 g, 8.3% batch weight) were agitated in a glass reactor. The mixture was then heated and held at 270° F. for 3 hours, whereupon the unreacted xylene in the alkylate mixture was less than 1.5%. The mixture was then cooled to 15° F. (65° C.) Agitation was stopped, and the mixture was allowed to stand for two hours. The catalyst, which separated from the alkylate product, was removed. GC analysis of the alkylate mixture showed that it contained unreacted o-xylene (1%), unreacted olefin (10%), branched monododecyl-o-xylene (85.5%), and didodecyl-o-xylenes (3.5%). Owing to their high degree of substitution and/or steric hindrance, the unreacted propylene tetramer isomers are virtually inert to the sulfonating agent. Therefore, their removal prior to the subsequent sulfonation of the alkylate mixture (to prepare anionic sulfonate surfactant) is unnecessary.

In view of the many changes and modifications that can be made without departing from principles underlying the invention, reference should be made to the appended claims for an understanding of the scope of the protection afforded the invention.

What is claimed is:

1. A process for mono-alkylating at least one monocyclic aromatic hydrocarbon comprising reacting the monocyclic aromatic hydrocarbon with at least one α-olefin having from 4 to 20 carbon atoms in the presence of an anhydrous alkane sulfonic acid at a temperature below about 280° F.

2. The process of claim 1 wherein the reaction temperature is in the range of from about 180° F. to about 280° F.

3. The process of claim 1 wherein the monocyclic aromatic hydrocarbon is selected from the group consisting of benzene, toluene, o-xylene, m-xylene, p-xylene, hemimellitene, pseudocumene, mesitylene, prehnitene, isodurene, pentamethylbenzene, ethylbenzene, n-propylbenzene, cumene, n-butylbenzene, isobutylbenzene, sec-butylbenzene, tert-butylbenzen, cymene, biphenyl, diphenylmethane, triphenylmethane, 1,2-diphenylethane, styrene, trans-stilbene, cis-stilbene, unsym-diphenylethylene, triphenylethylene, tetraphenylethylene, phenylacetylene, and diphenylacetylene.

4. The process of claim 3 wherein the monocyclic aromatic hydrocarbon is selected from the group consisting of benzene, toluene, o-xylene, m-xylene, p-xylene, and mixtures thereof.

5. The process of claim 4 wherein the monocyclic aromatic hydrocarbon is o-xylene.

6. The process of claim 1 wherein the α-olefin is selected from the group consisting of 1-decene, 1-dodecene, 1-tetradecene, 1-hexadecene, and 1-octadecene.

7. The process of claim 6 wherein the α-olefin is 1-dodecene.

8. The process of claim 1 wherein the alkyl moiety of the anhydrous alkane sulfonic acid is one of from one to four carbon atoms.

9. The process of claims 8 wherein the anhydrous alkane sulfonic acid is anhydrous methane sulfonic acid.

10. The process of claim 1 wherein the reaction between the the monocyclic aromatic hydrocarbon with an α-olefin is initiated at a temperature in the range of from about 180 to about 200° F.

11. The process of claim 10 wherein, after initiation, the reaction temperature is maintained at a temperature in the range of from about 250 to about 270° F. until alkylation is complete.

12. A process for mono-alkylating o-xylene comprising:
A) mixing o-xylene, 1-dodecene, and anhydrous methane sulfonic acid in a reaction vessel;
B) initiating a reaction between the o-xylene and 1-dodecene by heating the contents of the reaction vessel to a temperature in the range of from about 180 to about 200° F.; and
C) maintaining the contents of the reaction vessel, after initation, at a temperature in the range of from about 250 to about 270° F. until alkylation is complete.
